(19) United States
(12) Patent Application Publication
(10) Pub. No.: US 2026/0027168 A1
(43) Pub. Date: Jan. 29, 2026

(54) NUTRITIONAL SUPPLEMENTS AND METHODS OF NUTRITIONAL SUPPLEMENTATION AFFECTING MOOD

(71) Applicant: AMARE GLOBAL, Lehi, UT (US)

(72) Inventor: Shawn Talbott, Plymouth, MA (US)

(21) Appl. No.: 19/279,912

(22) Filed: Jul. 24, 2025

Related U.S. Application Data
(60) Provisional application No. 63/675,696, filed on Jul. 25, 2024.

(51) Int. Cl. A61K 35/747; A61K 31/19; A61K 31/716; A61K 36/074; A61K 36/63; A61K 36/87

(52) U.S. Cl. CPC A61K 35/747; A61K 31/19; A61K 31/716; A61K 36/074; A61K 36/63; A61K 36/87

(57) ABSTRACT

Nutritional supplements and methods of nutritional supplementation are described for improving a mood of a subject. The nutritional supplement includes a probiotic configured to lower symptoms of stress and anxiety, an alpha-glucan/beta-glucan blend configured to prime an immune system, short-chain fatty acids, and pomance or marc of a grape, olive, or fruit.

### CROSS REFERENCE TO RELATED APPLICATIONS

This application claims the benefit of U.S. Provisional Patent Application No. 63/675,696, filed 25 Jul. 2024, which is incorporated by reference herein in its entirety.

### BACKGROUND

The following relates generally to nutritional supplements and to methods of nutritional supplementation of subjects. More specifically, the following relates to nutritional supplements affecting mood and to methods of supplementation of subjects.

In recent years, research has compiled evidence that the gut microbiome of a subject may influence the communication between that subject's central and enteric nervous systems. This communication appears to travel both directions, and an imbalance of a subject's gut flora has been associated with a variety of complaints, ranging from minor discomforts to significant CNS and gastrointestinal disorders. It has been observed that a healthy gut microbiome may improve mental health and aid in weight loss. Additionally, research has compiled evidence that improving the gut barrier function (i.e., “reducing leaky gut”) can improve immune system function, leading in some instances to a lower incidence of upper respiratory tract infections and for some a reduction in allergy symptoms. This can enable the immune system to operate as a more efficient “communication organ” in carrying signals across the gut-brain-axis, so people feel better (better mood, higher energy, etc.).

There is thus a need for nutritional supplements and methods of nutritional supplementation to support the immune systems of a patient to support and improve the mood of patients in need thereof.

### SUMMARY

This application describes improved nutritional supplements and methods of nutritional supplementation that affect the gut-brain axis and/or affect mood. Generally, the described techniques provide for unique nutritional supplements and methods of their use to improve the mood of a subject, and to positively influence that subject's health, metabolism and mental health.

In some aspects, a nutritional supplement is described. The nutritional supplement includes a probiotic configured to lower symptoms of stress and anxiety, an alpha-glucan/beta-glucan blend configured to prime an immune system, short-chain fatty acids, and pomance or marc of a grape, olive, or fruit.

In some aspects, a method of improving a mood of a subject is described. The method may include administering to the subject an effective amount of a nutritional supplement including a probiotic configured to lower symptoms of stress and anxiety, an alpha-glucan/beta-glucan blend configured to prime an immune system, short-chain fatty acids, and pomance or marc of a grape, olive, or fruit.

### DETAILED DESCRIPTION

This application describes compositions useful as improved nutritional supplements and methods of their use in supplementing the nutrition of patients to improve mood and to provide positive changes in their health and metabolism.

Amounts, concentrations, and other numerical data may be presented herein in a

range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, an amount of from 1 mg to 200 mg should be interpreted to include not only the explicitly recited limits of 1 mg and about 200 mg, but also to include individual amounts such as 2 mg, 3 mg, 4 mg, and sub-ranges such as 10 mg to 50 mg, 20 mg to 100 mg, etc. Unless otherwise stated, all ranges include both endpoints.

As used herein, treatment means any manner in which the symptoms or pathology of a condition, disorder or disease are ameliorated or otherwise beneficially altered. Treatment also encompasses any pharmaceutical use of the compositions herein.

The supplement compositions of the present disclosure may be administered in a variety of suitable dosage forms, including, without limitation, tablets, capsules, granules, powders, liquids, liposome inclusions, ointments, gels, external powders, sprays, inhalable powders, injectable preparations (solutions, suspensions, emulsions, solids to be dissolved when used, etc.), eye drops, eye ointments, suppositories, and the like can be selected appropriately depending on the administration method, and the compositions of the present disclosure can be accordingly formulated. Formulation in general is described in references including Comprehensive Medicinal Chemistry, Volume 5, Editor Hansch et al, Pergamon Press 1990.

As used herein, “effective amount” refers to an amount of a substance which is sufficient to achieve its intended purpose or effect. Various biological factors may affect the ability of a delivered substance to perform its intended task. Therefore, an “effective amount” may be dependent on such biological factors. An effective amount of a compound for treating a disorder is an amount that is sufficient to ameliorate, or in some manner reduce a symptom or stop or reverse progression of a condition. Such amount may be administered as a single dosage or may be administered according to a regimen whereby it is effective. The achievement of therapeutic effects may be measured by a physician or other qualified medical personnel using evaluations known in the art, (for example with testosterone supplementation therapy, physical examination, blood and saliva tests may be used), it is recognized that individual variation and response to treatments may make the achievement of therapeutic effects a subjective decision.

As used herein, “administration,” and “administering” may be used interchangeably, and refer to the act of presenting, applying, or introducing a drug to a subject in order to achieve a desired physiological or psychological response.

As used herein, amelioration of the symptoms of a particular disorder by administration of a particular pharmaceutical composition refers to any lessening, whether permanent or temporary, lasting or transient that can be attributed to or associated with administration of the composition.

In some aspects, a nutritional supplement for improving mood and general health, of a subject is described. The nutritional supplement may include a plurality of prebiotics, probiotics, postbiotics, and phytobiotics that improve the mood of the subject. In an illustrated embodiment, the nutritional supplement includes at least four primary ingredients, and, in some embodiments, the nutritional supplement may include other primary ingredients. The primary ingredients are combined in specific ratios to synchronize with all parts of the gut-brain axis through prebiotics, probiotics, postbiotics, and phytobiotics to improve mental wellness and physical composition. Accordingly, the nutritional supplements described herein are natural mood enhancement products that operate through a synergistic 4-part process that automates, utilizes, and synchronizes all parts of the gut-brain axis through prebiotics, probiotics, postbiotics, and phytobiotics to improve mental wellness and physical composition.

In an exemplary embodiment, the nutritional supplement includes a first primary ingredient including a probiotic that lowers symptoms of stress and anxiety, as well as the biomarker cortisol. Specifically, in the exemplary embodiment, the probiotic includes Lactobacillus Plantarum. Lactobacillus Plantarum works via the gut-brain axis and has been shown to optimize the serotonin pathway and gut modulation. Lactobacillus Plantarum further influences the norepinephrine pathway with increased dopamine levels, improving mood. Lactobacillus Plantarum may modulate tryptophan-serotonin which could be one of several mechanisms involved demonstrating that Lactobacillus Plantarum lowers symptoms of stress and anxiety, as well as biomarker cortisol. In alternative embodiments, the nutritional supplement may include any probiotic that enables the nutritional supplement to operate as described herein.

In the exemplary embodiment, the nutritional supplement further includes a second primary ingredient including an alpha-glucan/beta-glucan blend that primes the immune system. Specifically, in the exemplary embodiment, the alpha-glucan/beta-glucan blend includes at least one mushroom (agaricus or agaricus bisporus). The at least one mushroom may include a plurality of mushrooms and may include any type of mushroom that enables the supplements and treatments described herein to operate as described herein.

In some embodiments, the mushrooms may any one or more of Reishi, Oyster, and/or Maitake mushrooms. Studies have shown that mushrooms prime the immune system and may help to restore the gut barrier and improve gut diversity. In alternative embodiments, the nutritional supplement may include any alpha-glucan/beta-glucan blend that enables the nutritional supplement to operate as described herein.

The exemplary embodiment of the nutritional supplement may further include a third primary ingredient including short-chain fatty acids (SCFAs). Specifically, in the exemplary embodiment, the SCFAs include butyrate but may also include any SCFAs that enable the nutritional supplements described herein to operate as described herein. Butyrate is produced when bacteria in the gut help break down dietary fiber in the large intestine (colon).

Studies have shown that Butyrate improves digestive health by providing the main energy source for colon cells. Additionally, butyrate may provide other health benefits including supporting the immune system, reducing inflammation, and preventing diseases. In alternative embodiments, the nutritional supplement may include any SCFA that enables the nutritional supplement to operate as described herein.

In the exemplary embodiment, the nutritional supplement also includes a fourth primary ingredient including pomance or marc. Specifically, in the exemplary embodiment, the pomance or marc includes chardonnay marc but may also include the pomance or marc of any grape, olive, and/or fruit that enables the nutritional supplements described herein to operate as described herein. Pomance or marc includes the solid remains of grapes, olives, or other fruit after pressing for juice or oil and contains the skins, pulp, seeds, and stems of the fruit. Studies have shown that pomance or marc is rich in prebiotic oligosaccharides and gut-brain-supporting flavonoids. Additionally, pomance or marc may provide other health benefits including supporting the immune system, reducing inflammation, and preventing diseases. In alternative embodiments, the nutritional supplement may include any pomance or marc that enables the nutritional supplement to operate as described herein.

In the exemplary embodiment, the nutritional supplements described herein include four primary ingredients. In some embodiments, the nutritional supplement may include five or more primary ingredients.

Studies have demonstrated that priming the immune system improves mental well-being. For example, the four primary ingredients described herein have been found to improve gut barrier function (i.e., “reducing leaky gut”) and improve immune system function (i.e., fewer upper respiratory tract infections and less allergy symptoms). Thus, the nutritional supplements described herein enable the immune system to be a more efficient “communication organ” in carrying signals across the gut-brain-axis, improving the mood and increasing the energy of the subject.

In the four primary ingredient embodiment, the nutritional supplement includes from about 500 million to about 2 billion Colony Forming Units (“CFUs”) of the anti-stress probiotic, from about 10 milligrams (“mg”) to about 100 mg of the alpha-glucan/beta-glucan blend, from about 10 milligrams (“mg”) to about 500 mg of SCFAs, and from about 1,000 mg to about 5,000 mg of pomance or marc. In alternative embodiments, the nutritional supplement may include any amount of the anti-stress probiotic, the alpha-glucan/beta-glucan blend, SCFAs, and the pomance or marc that enables the nutritional supplement to operate as described herein.

In some aspects, a method of improving the mood of a subject is described. The method may include administering to the subject an effective amount of a nutritional supplement including an anti-stress probiotic, an alpha-glucan/beta-glucan blend, a short-chain fatty acid, and pomance or marc.

As used herein, including in the claims, “or” as used in a list of items (e.g., a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

The description set forth herein, in connection with the example, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A nutritional supplement comprising:
a probiotic configured to lower symptoms of stress and anxiety;
an alpha-glucan/beta-glucan blend configured to prime an immune system;
short-chain fatty acids; and
pomance or marc of a grape, olive, or fruit.

2. The nutritional supplement of claim 1, wherein the probiotic comprises an anti-stress probiotic.

3. The nutritional supplement of claim 2, wherein the anti-stress probiotic comprises Lactobacillus Plantarum.

4. The nutritional supplement of claim 3, wherein the nutritional supplement comprises from about 500 million to about 2 billion Colony Forming Units of Lactobacillus Plantarum.

5. The nutritional supplement of claim 1, wherein the alpha-glucan/beta-glucan blend comprises at least one mushroom.

6. The nutritional supplement of claim 5, wherein the at least one mushroom comprises a plurality of mushrooms.

7. The nutritional supplement of claim 6, wherein the plurality of mushrooms comprises at least one of Reishi, Oyster, or Maitake mushrooms.

8. The nutritional supplement of claim 1, wherein the nutritional supplement comprises between about 10 milligrams (“mg”) and about 100 mg of the alpha-glucan/beta-glucan blend.

9. The nutritional supplement of claim 4, wherein the short-chain fatty acids comprise from about 10 mg to about 500 mg of butyrate.

10. The nutritional supplement of claim 1, wherein the pomance or marc of a grape, olive, or fruit comprises from about 1,000 mg to about 5,000 mg of chardonnay pomance or marc.

11. A method of improving a mood of a subject, the method comprising:
administering to the subject an effective amount of a nutritional supplement comprising:
a probiotic configured to lower symptoms of stress and anxiety;
an alpha-glucan/beta-glucan blend configured to prime an immune system;
short-chain fatty acids; and
pomance or marc of a grape, olive, or fruit.

12. The method of claim 11, wherein the probiotic comprises an anti-stress probiotic.

13. The method of claim 12, wherein the anti-stress probiotic comprises Lactobacillus Plantarum.

14. The method of claim 13, wherein the nutritional supplement comprises between about 500 million to about 2 billion Colony Forming Units of Lactobacillus Plantarum.

15. The method of claim 11, wherein the alpha-glucan/beta-glucan blend comprises at least one mushroom.

16. The method of claim 15, wherein the at least one mushroom comprises a plurality of mushrooms.

17. The method of claim 16, wherein the plurality of mushrooms comprises at least one of Reishi, Oyster, or Maitake mushrooms.

18. The method of claim 11, wherein the nutritional supplement comprises between about 10 milligrams (“mg”) and about 100 mg of the alpha-glucan/beta-glucan blend.

19. The method of claim 11, wherein the short-chain fatty acids comprise from about 10 mg to about 500 mg of butyrate.

20. The method of claim 11. wherein the pomance or mare of a grape, olive, or fruit comprises from about 1.000 mg to about 5.000 mg of chardonnay pomance or marc.
